(19) United States
(12) Patent Application Publication
(10) Pub. No.: US 2006/0264357 A1
(43) Pub. Date: Nov. 23, 2006

(54) Capillary membrane stabilization and reduction of tissue injury through use of biodegradable macromolecules with antioxidants and/or other chemicals

(76) Inventors: Bashir A. Zikria, Norwood, NJ (US); Jemal Dean Zikria, Old Greenwich, CT (US)

Correspondence Address: Evelyn M. Sommer, 250 Park Avenue, Room 825, New York, NY, 10022, US

(21) Appl. No.: 11/213,303

(22) Filed: Aug. 29, 2005

Related U.S. Application Data
(63) Continuation-in-part of application No. 08/837,840, filed on Apr. 22, 1997, now Pat. No. 7,041,655.

(51) Int. Cl. A61K 38/48; A61K 38/40; A61K 38/22; A61K 38/54; A61K 48/00; A61K 31/7076; A61K 31/716; A61K 31/44; A61K 31/4172; A61K 31/353; A61K 31/715; A61K 31/385; A61K 31/355

(52) U.S. Cl. 514/6; 514/12; 514/54; 514/59; 514/60; 514/44; 514/25; 514/27; 514/400; 514/456; 514/440; 514/458; 514/383; 514/358; 514/460; 514/47; 514/553; 514/763; 514/731; 514/651

(57) ABSTRACT

The present invention provides a method of treating a human subject to prevent leakage of serum proteins from capillary endothelial junctions during a period of increased capillary permeability and at the same time preventing the harmful effects of free radicals on capillaries and surrounding tissues. The method comprises administering to a subject an effective amount of a composition comprising at least one polysaccharide selected from the group of HES, glycogen and dextran of varying molecular sizes and at least one active agent selected from the group consisting of dehydroascorbic acid, von Willebrand Factor, hemoglobin, polysaccharide-conjugated hemoglobin, Cerovive, edaravone, dimethylthiourea, citicoline, poly(ADP-ribose) polymerase inhibitor, oxidant detoxification catalyst, adenosine 2a (A2a) receptor agonist, adenosine 1 (A1) receptor agonist, adenosine, inosine, xanthin oxidase inhibitor, polyethylene-glycol-modified albumin, adenosine triphosphate, histamine, taurine, simvastatin, atrial natriuretic peptide, sphinogosine 1-phosphate, apyrase, secretory leukocyte protease inhibitor, antithrombin III, adrenomedullin, intravenous immunologlobulin, sodium beta-aescin, Δ²-1,2,3-triazoline and aminoalkylpyridine, aromatase inhibitors, and neuropilin-1, polynitroxyl albumin, α-phenyl-N-tert-butyl nitrone and the antioxidant subgroup consisting of tocopherols, tocotrienols, carotenoids, minerals and mineral-containing organic compounds, polyphenols, lipoic acids, transition metal ion-binding proteins, melatonin, hormones, polyamines, tamoxifen and its metabolites and propofol. The composition may further contain at least one member of the group of superoxide dismutase, glutathione peroxidase, catalase, hydroxyethyl rutoside, cyclic adenosine monophosphate and vitamin C. The compositions contain the macromolecules in a molecular size and concentration adequate to effectively stabilize the capillary membrane. The stabilization effect is accompanied by a biophysical and biochemical process due to the adhesiveness and configuration of the macromolecules, and because of their size. The treatment is benign as the macromolecules and active agents are non-toxic and biodegradable.

This application is a continuation-in-part of copending application Ser. No. 08/837840 filed Apr. 22, 1997.

The present invention relates to a method for treating human subjects to prevent leakage of macromolecules from capillary endothelial junctions while simultaneously preventing damage to the capillaries and surrounding tissues due to the presence of toxins, released free radicals, etc. More particularly this invention relates to macromolecules and biochemical methods for preventing leakage of macromolecules from capillary endothelial junctions during a period of increased capillary permeability secondary to burn injury, battlefield injury, cytotoxicity, trauma, septic and hemorrhagic shock, ischemia and other toxic processes while simultaneously preventing pathology due to the activity of free radicals.

The compositions of the invention comprise at least one macromolecular polysaccharide selected from the group consisting of hetastarch (HES), glycogen, and dextran and at least one pharmacologically active agent comprising protective agents selected from citicoline, dehydroascorbic acid, von Willebrand Factor, hemoglobin, polysaccharide-conjugated hemoglobin, poly(ADP-ribose) polymerase inhibitor, oxidant detoxification catalyst, adenosine 2a (A2a) receptor agonist, adenosine 1 (A1) receptor agonist, adenosine, inosine, xanthin oxidase inhibitor, polyethylene-glycol-modified albumin, adenosine triphosphate, histamine, taurine, simvastatin, atrial natriuretic peptide, sphinogosine 1-phosphate, apyrase, secretory leukocyte protease inhibitor, antithrombin III, adrenomedullin, intravenous immunologlobulin, sodium beta-aescin, Δ²-1,2,3-triazoline and aminoalkylpyridine, aromatase inhibitors, and neuropilin-1, spin trapping compounds such as Cerovive®, polynitroxyl albumin, and PBN, free radical scavengers such as edaravone and dimethylthiourea, and antioxidant agents selected from tocopherols, tocotrienols, carotenoids, antioxidant minerals and active organic compounds containing such minerals, polyphenols, lipoic acids, transition metal ion binding proteins, melatonin, antioxidant hormones, polyamines, tamoxifen and propofol. Optionally the composition can further include at least one antioxidant agent selected from superoxide dismutase, glutathione peroxidase, catalase, hydroxyethyl rutoside, cyclic adenosine monophosphate and vitamin C.

The protective action of these polysaccharides macromolecules has been shown to be brought about by biophysical and biochemical processes resulting in membrane stabilization of the capillary endothelial cell by virtue of the “sealing” effects of these macromolecules and possibly their anti-inflammatory activities. The beneficial effects of spin trapping compounds are based on their capacity in neutralizing harmful free radicals. The protective effects of the antioxidants are due to their biochemical activity alone or in combination in neutralizing the harmful effects of free radicals. Citicoline's protective mechanism is still under study. Von Willebrand factor performs two essential functions in hemostasis, the mediation of the adhesion of platelets to subendothelial connective tissue, and the binding of blood clotting factor VIII. Hemoglobin and modified hemoglobin can be used as an essential oxygen-carrying composition in the preparation of a blood substitute.

In co-pending application Ser. No. 08/837840, there is disclosed a novel method for treating a human subject to prevent leakage of serum proteins from capillary endothelial junctions during a period of increased capillary permeability and at the same time, preventing the harmful effects of free radicals on cellular membranes and other organelles. The entirety of this co-pending application is incorporated herein by reference thereto. The method comprises administering to a subject an effective amount of a composition comprising at least one polysaccharide selected from hydroxyethyl starch and dextran of varying molecular size and at least one member of the antioxidant group consisting of superoxide dismutase, glutathione peroxidase, catalase, hydroxyethyl rutoside, cyclic adenosine monophosphate and vitamin C.

Antioxidants and other protective agents are essential components of the above-identified compositions having important biological functions in the composition proposed by the inventor.

Antioxidants are compounds that protect cells against the damaging effects of reactive oxygen species, such as singlet oxygen, superoxide, peroxyl radicals, hydroxyl radicals and peroxynitrite. An imbalance between antioxidants and reactive oxygen species results in oxidative stress, leading to cellular damage. The damage caused by free radicals to cell membranes can lead to fluid leakage and at the same time prevent the intake of cell nutrients. Free radicals interact with DNA and RNA resulting in the production of mutations and may also cause uncontrolled fusion of large cell molecules. Free radical pathology plays a part in immune system suppression and susceptibility to infectious diseases. Oxidative stress has been linked to a large number of pathological conditions, such as many central nervous system diseases including aging, stroke, Parkinsonism, Schizophrenia, Alzheimer's disease, Down Syndrome trauma, vascular headaches, cerebral palsy, diabetic neuropathy and neuroanesthesia adjunctcancer, peripheral nervous system diseases such as diabetic peripheral neuropathy and traumatic nerve damage, as well as peripheral organ diseases such as cancer, atherosclerosis, pulmonary fibrosis, pancreatitis, ischemic injury, inflammation, angioplasty, multiple organ failure, burns, decubitus ulcers, ischemic bowel disease, and age-related macular degeneration.

Antioxidant mechanisms of action include scavenging reactive oxygen and nitrogen free radicals, decreasing the localized oxygen concentration thereby reducing molecular oxygen's oxidation potential, metabolizing lipid peroxide to non-radical products, and chelating metal ions to prevent the generation of free radicals. By so doing, antioxidants limit the free radical damage resulting from oxidizing low density lipoprotein cholesterol, promoting platelet adhesion, damaging the cell's DNA, blocking the normal endothelial cell function and vasodilatation in response to nitric oxide, triggering inflammation and impairing immune function.

It has now been found that other antioxidants, than those previously disclosed used singly or in combination, can have similar beneficial effects on the human subject if administered in a similar manner together with the disclosed polysaccharide.

These antioxidants are selected from the following categories.
A. Tocopherols (such as vitamin E), tocotrienols and their derivatives such as acetates and succinates.
B. Carotenoids.
C. Minerals such as zinc, selenium and magnesium. Selenium-containing compounds further include selenoproteins.
D. Polyphenols.
E. Lipoic acids such as alpha-lipoic acid and dihydrolipoic acid.
F. Transition metal ion-binding proteins.
G. Melatonin.
H. Hormones and hormone-related compounds.
I. Polyamines
J. Tamoxifen and its metabolites
K. Propofol

The tocopherols and tocotrienols are known to possess potent antioxidant properties. The tocopherols include alpha-(Vitamin E), beta-, gamma- and delta-tocopherols. The tocotrienols also include alpha-, beta-, gamma- and delta-varieties.

Carotenoids are a large family of antioxidant compounds and some of the most abundant carotenoids include alpha-carotene, beta-carotene (also known as Vitamin A), gamma-carotene, lycopene, lutein, beta-crytoxanthin, zeaxanthin and astaxanthin. These compounds are readily available from such foods as carrots, pumpkins, avocados, red peppers, apricots, spinach, tomatoes, grapefruits, watermelons, kale and brussel sprouts. Beta-carotene, the precursor for vitamin A (retinol) is commonly recognized for its antioxidant properties and may be useful against the negative effect of oxidative damage, especially in the absence of alcohol (Schafer et al. Biol. Chem. 383 (3-4): 671-681 (2002)). The antioxidant activities of lycopene and lutein have also been recognized and synergistic effects between carotenoids, especially that between lycopene and lutein are known too. (Stahl et al. FEBS Lett. 427(2):305-308 (1998)).

Selenium has been identified as an essential nutrient for humans half a century ago. As of 2001, 15 selenoproteins have been studied and many of them, such as glutathione perioxidases are known as antioxidants. Recently other antioxidants have been identified in the category of selenium containing compounds, such as selenoprotein P.

Zinc can also act as a free radical scavenger. Zinc deficiency has been shown in animal research to correlate with an increase in lipid peroxidation, which is remediable with zinc supplementation (Ozturk et al. Biol. Trace Elem. Res. 94(2):157-166 (2003)).

Magnesium is also known to possess antioxidant activities.

There are many types of naturally occurring antioxidant polyphenols. Flavonoids are a type of polyphenol whose examples include anthocyanins, flavones, flavonols (such as quercetin, rutin and catechins), flavanones and isoflavones. Other types of antioxidant polyphenols include phenolic acids such as hydroxybenzoic acids examples of which include gallic acids, ellagic acid, salicylic acid, and non-acid phenolic compounds such as capsaicin and tannins.

Flavonoids have shown multiple biological benefits in human consumption. Epidemiological studies have shown that flavonoid intake is inversely related to the mortality from coronary heart diseases and to the incidence of heart attacks. (U.S. Pat. No. 6,818,233 and Hertog et al. The Lancet, 342(8878):1007-1011 (1993)). Flavonoids have also been shown to effectively protect low-density lipoprotein from Cu²⁺-catalyzed oxidation and thus reduce the likelihood of atherosclerosis. (Miranda et al. J. Agric. Food Chem. 48: 3876-3884 (2000)). Flavonoids are known to act similarly to vitamin E in the protection of biomembranes from free-radical induced tissue damages. (van Acker et al. FEBS. Lett. 473: 145-148 (2000)).

The capacity of flavonoids to act as antioxidants depends on their molecular structure. The position of hydroxyl groups and other features in the chemical structure of flavonoids are important for their antioxidant and free radical scavenging activities. Examples of suitable antioxidant flavonoids include, in the order of decreasing potency, quercetin, xanthohumol, isoxanthohumol, and genistein.

Lipoic acids, such as alpha-lipoic acid (also known as thioctic acid) are known to function as antioxidants. Antioxidant powers of alpha-lipoic acids have been documented in animal and humans. (Wollin et al. J. Nutr. 133(11):3327-3330 (2003) and J. Gerontol. A Biol. Sci. Med. Sci. 58 (9):B788-791 (2003)). Alpha-dihydrolipoic acid, the reduced form of alpha-lipoic acid, is the only form that functions directly as an antioxidant. Dihydrolipoic acid can directly terminate free radicals by scavenging reactive oxygen species and reactive nitrogen species, chelate transition metal ions, thereby reducing their harmful reactivity, increase intracellular glutathione levels, repair oxidative damage, and regenerate other antioxidants such as vitamin C, glutathione, coenzyme Q10 and alpha-tocopherol (vitamin E). Alpha-lipoic acid is also capable of chelating metal ions such as iron and copper. (Biewenga et al. Gen. Pharmac. 29(3):315-331 (1997) and Smith et al. Curr. Med. Chem. 11(9):1135-1146 (2004)).

Although iron is an essential element in the human, uncontrolled ferrous ions promote lipid peroxidation and oxidative DNA damage, since iron is involved in the formation of hydroxyl radicals (OH). A few other transition metal ions, such as copper, could bring about similar oxidative damage. Therefore, transition metal ion-binding proteins, which can reduce the effective concentration of these transition metal ions, can lead to reduced oxidative activities and thus function as antioxidants.

The antioxidant properties of ceruloplasmin has been established recently (Atanasiu et al. Mol. Cell. Biochem. 189:127-135 (1998)). Ceruloplasmin, a plasma protein, is a multifunctional α₂-globulin involved in transporting 95% of the copper in blood. Ferroxidase activities are also observed with ceruloplasmin. It has been shown to be more effective as a peroxyl radical scavenger than superoxide dismutase, deferoxamine and bovine serum albumin, but slightly less effective than catalase. Heat-denatured ceruloplasmin was shown to possess higher potency than regular caeruplasmin.

Deferoxamine, an iron chelator, is known to be an antioxidant and acts by preventing ferrous ions from promoting peroxidation and oxidative DNA damage. Melatonin, 5-methoxytryptophol or pinoline have been shown to enhance the efficacy of deferoxamine in preventing lipid peroxidation (Ortega-Gutierrez et al. Neurosci. Lett. 323:55-59 (2002)).

Lactoferrin, a natural defense iron-binding protein is known to scavenge non-protein-bound iron in body fluids and inflamed areas so as to suppress free radical mediated damage (Weinberg et al. Expert Opin. Investig. Drugs 12(5):841-851 (2003)).

Transferrin, present principally in serum, is also a iron transport protein which acts as an antioxidant by reducing the concentration of free ferrous ion (Chauhan et al. Life Sci. 75:2539-2549 (2004)).

Melatonin has been observed to possess potent antioxidant properties by acting directly as a free radical detoxifying agent, or indirectly by changing the activity of enzymes that metabolize active oxygen species to inactive products. It has been shown to inhibit the prooxidative enzyme nitric oxide synthase, stimulate the activity of several antioxidative enzymes, and prevent membrane fluidity changes associated with peroxidation. (Reiter et al. Drug News Perspect. 11(5): 291-296 (1998)).

Several hormones also possess antioxidant properties. Estrogens, such as estrone, estradiol and estriol have been found to possess substantial activities with respect to the inhibition of lipid peroxidation (Sugioka et al. FEBS 210(1):37-39 (1987)). Thyroxine has been found to possess a potent antioxidant activity on iron-induced phospholipids peroxidation (Suwa et al. Proc. Soc. Exp. Biol. Med. 150:401-406 (1975)). Dehydroepiandrosterone (DHEA), a precursor for steroid sex hormones produced in adrenals and gonads, is also recognized as an antioxidant, as well as are its derivatives such as 7-alpha-hydroxy-DHEA. (Pelissier et al. Steroids 69: 137-144 (2004); Brignardello et al. J. Endocrinol. 166: 401-406 (2000)). In short-term animal tests, DHEA was shown to be able to compensate for vitamin E deficiency in vivo. (Ng et al. Food Chem. Toxicol. 37: 503-508 (1999)).

Polyamines, whose examples include cadaverine, putrescine, spermidine and spermine, have been shown to possess antioxidant capacities, such as scavenging radicals, decreasing lipid peroxidation and protect DNA from oxidative damages. (Das et al. Mol. Cell Biochem. 262 (1-2):127-133 (2004)).

Tamoxifen and its metabolites such as 4-hydroxytamoxifen are also known to possess antioxidant properties, such as inhibition of lipid peroxidation. (Wiseman et al. FEBS Lett. 26392):192-194 (1990)).

Propofol also possesses antioxidant properties and has been shown to attenuate lung endothelial injury induced by ischemia-reperfusion and oxidative stress (Balyasnikova et al. Anesth. Analg. 100:929-936 (2005)).

Other antioxidants that can be used in the compositions of the invention include albumin, terpenoids, organosulfur compounds, indoles, lignans, coenzyme Q, uric acid, copper, and pycnogenol. Creatine has also been shown to exhibit direct antioxidant properties, capable of neutralizing radical and reactive species that are aqueous ions (Lawler et al. Biochem. Biophys. Res. Commun. 290:47-52 (2002)).

The antioxidants may be used in the final compositions individually or in combinations. Synergy between some of the antioxidants has been observed, such as that between alpha-tocopherol and the carotenoid zeaxanthin in protecting liposomes against lipid peroxidation, while alpha-tocopherol alone failing to show a protective effect (Wrona et al. Free Radic. Biol. Med. 35(10):1319-1329 (2003)), that between vitamin E and a mixture of carotenoids (Upritchard et al. Am. J. Clin. Nutr. 78(5): 985-992 (2003)), that between vitamin E and flavonoids (U.S. Pat. No. 6,251,400) and others. Significant synergistic effects were observed in mixtures of carotenoids, especially when lycopene or lutein was present (Stahl et al. FEBS Lett. 427(2): 305-308 (1998)).

Citicoline, an exogenous form of cytidine-5′-diphosphocholine, has been suggested to possess versatile neuroprotective properties for central nervous system injuries and neurodegenerative disorders, such as cerebral ischemia, with virtually no observed side-effects (Adibhatla et al. J. Neurochem. 80:12-23 (2002)). Citicoline may reduce ischemic injury by stabilizing membranes and decreasing free radical formations. Citicoline has been shown to decrease lipid peroxidation following transient cerebral ischemia (Fresta et al. J. Pharm. Pharmacol. 46:974-981 (1994)). Citicoline is also shown to lead to an increased level of glutathione which may attenuate lipid peroxidation (Rao et al. J. Neurochem. 75:2528-2535 (2000)).

Spin trapping compounds and free radical scavengers have been discovered to be effective in treating a variety of disorders, including disorders such as those arising from ischemia, infection, inflammation, exposure to radiation or cytotoxic compounds, not just of the central and peripheral nervous systems but of peripheral organ disease having a wide variety of etiology (U.S. Pat. No. 6,403,627). Examples of stable spin trapping compounds are various nitroxides and nitrones, such as those disclosed in U.S. Pat. Nos. 6,403,627, 5,750,710, 5,723,502, and Zhang et al. Free Radic. Biol. Med. 29:42-50 (2000). Examples of free radical scavengers include edaravone and dimethylthiourea. They, unlike other antioxidants, generally neither act as proxidants, nor do they propagate free radical chain reactions. While all non-toxic and stable spin trapping compounds and free radical scavengers can be used in the formulation, the preferred agents are PBN (α-phenyl-N-tert-butyl nitrone), polynitroxyl albumin, Cerovive® (NXY-059, disodium 4-[(tert-butylimino)-methyl]benzene-1,3-disulfonate N-oxide), edaravone and dimethylthiourea, which possess well-documented neuroprotective properties during ischemia (Marshall et al. Stroke 32:190-198 (2001); Kawai et al. J. Pharmcol. Exp. Ther. 281:921-927 (1997); Gutman et al. Cancer Immunol. Immunother. 43:240-244 (1996)).

Hemoglobin and in particular, modified hemoglobin, have been used as an essential component for blood substitute due to its ability in carrying oxygen (U.S. Pat. No. 6,844,317). The chemical modification is generally one of intramolecular cross-linking, oligomerization and/or polymer conjugation to modify the hemoglobin such that its persistence in the circulation is prolonged relative to that of unmodified hemoglobin, and its oxygen binding properties are similar to those of blood. Of particular relevance to this invention is hemoglobins conjugated to HES, dextran and glycogen.

Dehydroascorbic acid, the oxidized form of ascorbic acid (the antioxidant commonly known as Vitamin C), has been found to readily enter cells, in particular the brain cells, and be retained in the brain tissue in the form of ascorbic acid. Therefore, dehydroascorbic acid has been indicated for increasing the antioxidant potential of brain tissue of a subject (U.S. Pat. No. 6,608,106 and Agus et al. J. Clin. Invest. 100:2842-2848 (1997)).

Von Willebrand factor (VWF) is a blood glycoprotein that is required for normal hemostasis (Sadler Annu. Rev. Biochem. 67:395-424 (1998)). Deficiency of VWF, or von Willebrand Disease, is the most common inherited bleeding disorder. VWF mediates the adhesion of platelets to sites of vascular damage by binding to specific platelet membrane glycoproteins and to constituents of exposed connective tissue. VWF is also a carrier protein for blood clotting factor VIII. In the absence of VWF, factor VIII is rapidly removed from the circulation. Consequently, patients who lack VWF have a severe bleeding disorder because they have profound defects both in blood clotting and in the formation of platelet plugs at sites of vascular injury.

The activation of poly(ADP-ribose) polymerase (PARP) after exposure to nitric oxide or oxygen-free radicals can lead to cell injury via severe, irreversible depletion of the coenzyme nicotinamide adenine dinucleotide (NAD). In addition, PARP plays a central role in the caspase-independent apoptosis pathway mediated by apoptosis-inducing factor. Pharmacological inhibition of PARP has been shown to attenuate brain injury after focal ischemia, traumatic brain injury, Parkinson's disease and neurotoxicity in several neurodegeneratary models in animals (Iwashita et al. J. Pharmacol. Exp. Ther. 310: 425-436 (2004) and references cited therein). PARP inhibitors, such as 3-aminobenzamide (Park et al. Neurol. Res. 23:410-416 (2001)), N-(6-oxo-5,6-dihydro-phenanthridin-2-yl)-N,N-dimethylactamide, and 5-chloro-2-[3-(4-phenyl-3,6-dihydro-1(2H)-pyridinyl)propyl]-4(3H)-quinazolinone, have been shown to be effective in attenuating neuronal damages caused by the activation of PARP. In addition, PARP is required for efficient DNA repair and PARP activation rescues tumor cells from therapeutical DNA damage induced by chemotherapy agents. In the absence of PARP activity and the abse of homologous recombination repair, DNA double strand breaks remain unrepaired and lead to growth arrest and death of dividing tumor cell population. PARP inhibitors prevent tumor resistance to chemotherapy agents and restore susceptibility of tumors to chemotherapy. Second generation poly(ADP-ribose) polymerase inhibitors, such as INO-1002 (Inotek Pharmaceutical), have been known to useful in supportive care for prostate cancer.

A new class of metalloporphorynic compounds have been known with broad and potent activity against oxidative cell damage. WW-85, an oxidant detoxification catalyst, developed by Inotek Pharmaceuticals, is an extremely fast antioxidant catalyst with reaction rates of 5×10⁷ mol sec-1 in the degradation of peroxynitrite (to benign species of nitrite and nitrate) as well as hydrogen peroxide and nitroxyl anion. The catalytic nature allows WW-85 to be regenerated and delivered at very low therapeutic doses.

Adenosine 2a (A2a) receptor stimulation is anti-inflammatory as the receptors are used to sense excessive tissue inflammation. A2a receptor agonists, such as PJ-1165 (Inotek Pharmaceuticals), is known to be useful for suppressing inflammation.

Adenosine 1 (A1) receptor stimulation decreases AV nodal conduction and is thus useful as a means of treating atrial tachycardias without the side effects of calcium channel blockers, beta-blockers, and amiodarone. A1 receptor agonists, such as PJ-875 (Inotek Pharmaceuticals) is known to be useful in the emergency room and intensive care unit settings as a negative chronotropic agent for atrial tachycardia.

Adenosine is known to exert potent anti-inflammatory effects. For instance, adenosine has been shown to reduce the production of proinflammatory cytokines by inflammatory and noninflammatory cells stimulated by bacterial lipopolysaccharide (Hasko et al. J. Immunol. 157: 4634-4640 (1996); Sajjadi et al. J. Immunol. 156: 3435-3442 (1996); Wagner et al. Circ. Res. 82:47-56 (1998)).

Inosine, a endogenerous purine from the breakdown of adenosine, is known to effective in reducing systemic inflammation and improving survival in septic shock and other related disease states (Liaudet et al. Am. J. Respir. Crit. Care Med. 164:1213-1220 (2001)). It is believed that inosine functions for these intended benefits by potently inhibiting the release of proinflammatory cytokines and chemokines. Inosine analogues, such as IMS (Inotek Pharmaceuticals), offer similar benefits.

Xanthin oxidase (XO) is an enzyme that mediates the generation of superoxide anion in the myocardium in the setting of congestive heart failure. XO inhibitors, such as allopurinol and oxypurinol, have demonstrated remarkable efficacy in increasing contractility in congestive heart failure settings.

Polyethylene-glycol-modified albumin (PEG-Alb) is known to be a useful agent for plasma expansion during a period of capillary leak and hemodynamic compromise under systemic inflammatory response conditions (Assaly et al. Clin. Sci. (Lond), 107:263-272 (2004)). PEG-Alb can be retained in blood vessels whereas albumin extravasates into the interstitial space.

Endothelial barrier dysfunction caused by inflammatory agonists is a frequent underlying cause of vascular leak and edema. Adenosine Triphosphate (ATP) and its nonhydrolyzed analogs have been shown to be protective agents for the endothelial cell barrier and cause remodeling of cell-cell junctions (Kolosova et al. Circ. Res. 97:115-124 (2005)).

The therapeutic efficacy in the treatment of metastatic cancer with high doses of interleukin-2 has been limited by the onset of vascular leak syndrome and related toxicities. Histamine has been shown to improve survival and protects against interleukin-2 induced pulmonary vascular leak syndrome in animal models (Hornyak et al. Vascul. Pharmacol. 42:187-193 (2005)).

Taurine has been shown to decrease the endothelial injuries caused by interleukin-2 in acute lung injuries (Abdih et al. Eur. Surg. Res. 32:347-352 (2000)). It is believed that taurine acts in this regard in part by decreasing neutrophil-endothelial interactions.

Simvastatin, a 3-hydroxy-3-methylglutaryl (HMG)-CoA inhibitor, has been shown to attenuate vascular leak and inflammation in murine inflammatory lung injury (Jacobson et al. Am. J. Physiol. Lung Cell Mol. Physiol. 288:L1026-1032 (2005)).

Atrial natriuretic peptide (ANP) is known to reduce hypoxia-induced pulmonary vascular leak in vivo and protect endothelial barrier functions through its vasodilatory, natriuretic and other actions (Irwin et al. J. Physiol. Lung Cell Mol. Physiol. 288:L849-859 (2005)).

Gram-negative bacterial endotoxemia may lead to the pathological increase of vascular permeability with systemic vascular collapse, a vascular leak syndrome, multiple organ failure and/or shock. C1 inhibitor (C1INH) has been shown to provide certain benefits including the prevention of gram-negative bacterial lipopolysaccharide-induced vascular permeability (Liu et al. Blood 105:2350-2355 (2005)).

Sphinogosine 1-phosphate, a phospholipids angiogenic factor, has been shown to produce endothelial cell barrier enhancement and reduce vascular leak in murine and canine models of acute lung injury (McVerry et al. Am. J. Respir. Crit. Care Med. 170:987-993 (2004)).

Apyrase, a soluble NTPDase, has been shown to maintain vascular integrity and attenuate intestinal ischemia in animal models (Guckelberger et al. Thromb Haemost. 91:576-586 (2004)).

The secretory leukocyte protease inhibitor (SLPI) is found in a variety of secreted fluids in mammals and has been shown to suppress vascular permeability and provide anti-inflammatory effects (Mulligen et al. Am. J. Pathol. 156:1033-1039 (2000)).

Antithrombin III has been shown to attenuate tissue damage after local ischemia-reperfusion in several organ systems, including pulmonary injury (Aytekin et al. Am. J. Surg. 189:161-166 (2005)).

Adrenomedullin has also been known for the reduction of vascular leakage in sepsis and adult respiratory distress syndrome through actions including stabilization of the barrier function by cAMP-dependent relaxation of the microfilament system (Hippenstiel et al. Circ. Res. 91:618-625 (2002)).

Intravenous immunologlobulin has been found useful in the treatment of various clinical entities including protecting against mesenteric ischemia-reperfusion-induced local and remote injury (Anderson et al. Clin. Immunol. 114:137-146 (2005)).

Sodium beta-aescin is known to reduce the volume of cerebral infarct and water content and ameliorate the neurological deficit during ischemia-reperfusion injuries (Hu et al. Yao Xue Xue Bao 39:419-423 (2004)).

Δ²-1,2,3-triazoline and aminoalkylpyridine are known to be antiischemic and useful in the treatment of cerebral ischemia resulting from stroke (U.S. Pat. No. 6,638,954).

Other chemicals which can be added for various benefits include aromatase inhibitors, and neuropilin-1.

Hydroxyethyl starch (Hespan U.S. Pat. No. 3,523,938) is an artificial colloid derived from a waxy starch, composed almost entirely of amylopectin. The colloidal properties of 6% hetastarch approximate those of human albumin. Intravenous infusion of Hespan (hetastarch) results in expansion of the plasma volume slightly in excess of the volume infused but which decreases over the succeeding 24-36 hours. Hetastarch molecules below 50,000 daltons are rapidly eliminated by renal excretion with approximately 40% of a given total dose appearing in the urine in 24 hours.

HES is administered by intravenous infusion only. In adults the amount usually administered is 30 to 100 grams in solution. Doses of 1500 mls of hetastarch per day per 70 kg man have been used in postoperative and trauma patients. Hetastarch can be delivered in 0.9% saline, 5% dextrose or Ringer's lactate.

In accordance with the invention, polysaccharides other than hetastarch have produced promising results. These polysaccharide macromolecules include glycogen and dextran. Useful molecular weights for plasma substitution range from 100,000 to 500,000 Daltons. Dextran of appropriate molecular size does not pass through the capillary pores and therefore, can replace plasma proteins as colloid osmotic agents.

Few toxic reactions have been observed when using glycogen, dextran or hetastarch for fluid replacement therapy.

Methods for treating the above-identified conditions with the compositions of the invention comprising one or more polysaccharides containing in addition at least one pharmacological agent known to reduce the effects of trauma, inflammatory reactions, damage to endothelial cells and other tissues as well as agents known to enhance microcirculatory dynamics and reduce capillary permeability are an essential aspect of the invention. The pharmacologically active agents include dehydroascorbic acid, von Willebrand Factor, hemoglobin, polysaccharide-conjugated hemoglobin, citicoline, poly(ADP-ribose) polymerase inhibitor, oxidant detoxification catalyst, adenosine 2a (A2a) receptor agonist, adenosine 1 (A1) receptor agonist, adenosine, inosine, xanthin oxidase inhibitor, polyethylene-glycol-modified albumin, adenosine triphosphate, histamine, taurine, simvastatin, atrial natriuretic peptide, sphinogosine 1-phosphate, apyrase, secretory leukocyte protease inhibitor, antithrombin III, adrenomedullin, intravenous immunologlobulin, sodium beta-aescin, Δ²-1,2,3-triazoline and aminoalkylpyridine, aromatase inhibitors, and neuropilin-1, compounds possessing spin trapping properties such as PBN, polynitroxyl albumin, and Cerovive, compounds possessing free radical scavenging properties such as edaravone and dimethylthiourea, and compounds possessing antioxidant properties, such as tocopherols, tocotrienols, carotenoids, some minerals and mineral-containing organic compounds, polyphenols, lipoic acids, transition metal ion-binding proteins, melatonin, some hormones, polyamines, tamoxifen and its metabolites and propofol.

Compositions comprising a single polysaccharide macromolecule (HES, glycogen or dextran) or any two or all three together in combination with one or more active agents selected from the group of antioxidants, citicoline, von Willebrand Factor, hemoglobin, polysaccharide-conjugated hemoglobin, spin trapping compounds and free radical scavengers have been prepared for administration to human subjects who could benefit from the use to prevent leakage of serum protein from capillary endothelial junctions during period of increased capillary permeability. The compositions further comprising hydroxyethyl rutoside have been additionally prepared and used.

The following compositions of polysaccharide macromolecules and active agents on being prepared and administered can be administered and utilized in order to inhibit or prevent capillary leakage and inflammatory changes before the trauma incident was induced, for example extensive/major surgery or to correct or reduce the leakage after the trauma has occurred. The procedures as carried out, as for example described in application Ser. No. 837840 have shown the following: reduction of the pathological effects of trauma, reduction of the inflammatory reaction, reduction of damage to endothelial cells and other tissues, and enhancement of microcirculatory dynamics. These effects are realized as a consequence of the biophysical and biochemical properties of these molecules as capillary endothelial cell stabilizers by positively effecting the osmotic balance between the intra (capillary) and extra vascular space (interstitium), by preventing the adverse effect of free radicals and other biochemical pathways.

These findings have been recognized and appreciated and have been applied to the treatment of central nervous system injury, such as stroke, ruptured aneurysm, brain injury, and spinal cord injury etc., to major trauma such as surgery, limb salvage, burn injuries, poisonings (drug or snake venoms), to conditions such as pancreatitis, massive transfusions, anaphylaxis, sepsis, shock syndromes etc.

In addition to the conditions previously indicated, the compositions of this invention provide similar therapeutic benefits to patients with the following diseases and conditions, HIV/AIDS, hemorrhagic fever, avian flu, SARS, explosion injuries, lung injury (such as those as a result of explosion, smoke inhalation, battlefield, and chemical/biological warfare), extremities compartment syndrome, transplantation (for recipient and donor), organ preservation, cardioplegia, extracorporeal membrane oxygenation, plasma-pheresis, disease requiring renal dialysis, veno-venous (or arterio-venous) continuous ultra-filtration, abdominal compartment syndrome (intra-peritoneal hypertension), cancer chemotherapy and adult respiratory distress syndrome.

The total molecular weight range and composition of the final product may differ due to the fact that these macromolecules characteristically exhibit a wide range of molecular weights. The molecular weight ranges of the macromolecules used may also vary depending on the specific clinical application. For example, in compositions for use in treating cerebral trauma, a lower molecular range may be effective because the endothelial junctions of brain capillaries are considerably smaller than those in capillaries in other tissues.

Solutions of the macromolecules are prepared in 0.9 saline, 5% dextrose or Ringer's lactate, Ringer's lactate is the preferred carrier. The amount of the polysaccharide macromolecules in the compositions may vary but essentially range between 2-30%. The exact volume to be introduced intravenously is dependent on the specific clinical entity to be treated and the body weight of the subject. The usual volume is about 50 to 100 mls (2-30 grams in solution). However 1500 mls of 6% HES (9 grams) can readily be given to a 70 kg man over a 24 hour period. When the macromolecules are used in combination the total volume infused is similar to that used for a single macromolecule. The sum of the weight of macromolecules in the composition would be in the range of 3-25% and preferably between 5-15%, that is a total of 3-6 grams per 50 mls. Thus the composition would contain 1.5-3.0 grams of each of the component macromolecules per 50 mls if two are used in a 1:1 mixture. If used in a 4:1 mixture HES would be used as a 2.4-4.8 grams to 0.6-1.2 grams of dextran per 50 mls.

Solutions of the polysaccharide molecules are made up in either 0.9% saline, 5% dextrose or Ringer's lactate. The usual volume given by intravenous injection is 50 to 100 mls and contains about 5 to 15% polysaccharide macromolecules. For musculo-skeletal indications the HES would be of a molecular size ranging from 300 to 750 kD (kilo Daltons). Dextran would be used with an average molecular weight of 500 kD. Compositions for central nervous system treatment would contain HES with a molecular weight range of 150 to 400 kD. Dextran would be used with an average molecular weight of about 150 kD. Compositions for gastrointestinal pathology would use macromolecules of greater molecular size, hetastarch 500 to 1,000 kD and dextran 500 to 700 kD. The amount of a single macromolecule when used would be 3-6 grams per 50 mls and the molecular size utilized would depend on the clinical condition dictating its use. The following are representative molecular distributions of the HES portion of the formulations for different diseases states.
i. For disease states with tight capillary pores (such as brain injury, stroke, etc.)
   1. Weight average molecular weight: 150-210 kD.
   2. Number average molecular weight: 70-130 kD.
   3. Lower 10% no lower than: 50 kD.
   4. Upper 10% no greater than 350 kD.
   5. Substitution Ratio: 0.4-0.6.
ii. For disease states with general capillary leak (major trauma, sepsis, reperfusion injury, and most other similar afflictions, such as battlefield injury, cancer chemotherapy, shock syndrome and low flow states)
   1. Weight average molecular weight: 250-310 kD.
   2. Number average molecular weight: 170-230 kD.
   3. Lower 10% no lower than: 150 kD.
   4. Upper 10% no greater than: 450 kD.
   5. Substitution Ratio: 0.4-0.6.
iii. For disease states with capillary leak taking place with large capillary pores/leakage (such as lung injury)
   1. weight average molecular weight: 350-410 kD.
   2. Number average molecular weight: 270-330 kD.
   3. Lower 10% no lower than: 250 kD.
   4. Upper 10% no greater than: 550 kD.
   5. Substitution Ratio: 0.4-0.6.

The antioxidants and/or other protective agents would be added in the following concentrations expressed in the units of IU/kg per treatment, Units/ml per treatment, milimols/ml per treatment, mgms/ml per treatment.

### COMPOSITIONS

HES with hemoglobin
Glycogen with hemoglobin
Dextran with hemoglobin
HES, glycogen and dextran with hemoglobin
HES with HES-conjugated hemoglobin
Glycogen with glycogen-conjugated hemoglobin
Dextran with dextran-conjugated hemoglobin
HES with dehydroascorbic acid
Glycogen with dehydroascorbic acid
Dextran with dehydroascorbic acid
HES, glycogen and dextran with dehydroascorbic acid
HES with von Willebrand factor
Glycogen with von Willebrand factor
Dextran with von Willebrand factor
HES, glycogen and dextran with von Willebrand factor
HES with citicoline
Glycogen with citicoline
Dextran with citicoline
HES, glycogen and dextran with citicoline
HES with PBN
Glycogen with PBN
Dextran with PBN
HES, glycogen and dextran with PBN
HES with Cerovive
Glycogen with Cerovive
Dextran with Cerovive
HES, glycogen and dextran with Cerovive
HES with a tocopherol (15 IU)
Glycogen with a tocopherol (15 IU)
Dextran with a tocopherol (15 IU)
HES, glycogen and dextran with a tocopherol (15 IU)
HES with a tocotrienol (30-50 mg)
Glycogen with a tocotrienol (30-50 mg)
Dextran with a tocotrienol (30-50 mg)
HES, glycogen and dextran with a tocotrienol (30-50 mg)
HES with a carotenoid (25000 IU vitamin A activity)
Glycogen with a carotenoid (25000 IU vitamin A activity)
Dextran with a carotenoid (25000 IU vitamin A activity)
HES, glycogen and dextran with a carotenoid (25000 IU vitamin A activity)
HES with zinc (12-50 mg, preferably 12-15 mg)
Glycogen with zinc (12-50 mg, preferably 12-15 mg)
Dextran with zinc (12-50 mg, preferably 12-15 mg)
HES, glycogen and dextran with zinc (12-50 mg, preferably 12-15 mg)
HES with magnesium (300-500 mg, preferably 350 mg)
Glycogen with magnesium (300-500 mg, preferably 350 mg)
Dextran with magnesium (300-500 mg, preferably 350 mg)
HES, glycogen and dextran with magnesium (300-500 mg, preferably 350 mg)
HES with selenium (55-200 mcg, preferably 55-70 mg)
Glycogen with selenium (55-200 mcg, preferably 55-70 mg)
Dextran with selenium (55-200 mcg, preferably 55-70 mg)
HES, glycogen and dextran with selenium (55-200 mcg, preferably 55-70 mg)
HES with selenoprotein
Glycogen with selenoprotein
Dextran with selenoprotein
HES, glycogen and dextran with selenoprotein
HES with a flavonoid
Glycogen with a flavonoid
Dextran with a flavonoid
HES, glycogen and dextran with a flavonoid
HES with a phenolic acid
Glycogen with a phenolic acid
Dextran with a phenolic acid
HES, glycogen and dextran with a phenolic acid
HES with capsaicin (12.5 mg)
Glycogen with capsaicin (12.5 mg)
Dextran with capsaicin (12.5 mg)
HES, glycogen and dextran with capsaicin (12.5 mg)
HES with tannins
Glycogen with tannins
Dextran with tannins
HES, glycogen and dextran with tannins
HES with alpha-lipoic acid (100 mg BID)
Glycogen with alpha-lipoic acid (100 mg BID)
Dextran with alpha-lipoic acid (100 mg BID)
HES, glycogen and dextran with alpha-lipoic acid (100 mg BID)
HES with dihydrolipoic acid
Glycogen with dihydrolipoic acid
Dextran with dihydrolipoic acid
HES, glycogen and dextran with dihydrolipoic acid
HES with a transition metal ion-binding protein
Glycogen with a transition metal ion-binding protein
Dextran with a transition metal ion-binding protein
HES, glycogen and dextran with a transition metal ion-binding protein
HES with melatonin (1.2-3.0 mg)
Glycogen with melatonin (1.2-3.0 mg)
Dextran with melatonin (1.2-3.0 mg)
HES, glycogen and dextran with melatonin (1.2-3.0 mg)
HES with an estrogen
Glycogen with an estrogen
Dextran with an estrogen
HES, glycogen and dextran with an estrogen
HES with thyroxine
Glycogen with thyroxine
Dextran with thyroxine
HES, glycogen and dextran with thyroxine
HES with dehydroepiandrosterone (50 mg maximum per day for male, 25 mg maximum per day for female)
Glycogen with dehydroepiandrosterone (50 mg maximum per day for male, 25 mg maximum per day for female)
Dextran with dehydroepiandrosterone (50 mg maximum per day for male, 25 mg maximum per day for female)
HES, glycogen and dextran with dehydroepiandrosterone (50 mg maximum per day for male, 25 mg maximum per day for female)
HES with a polyamine
Glycogen with a polyamine
Dextran with a polyamine
HES, glycogen and dextran with a polyamine
HES with tamoxifen (20-40 mg)
Glycogen with tamoxifen (20-40 mg)
Dextran with tamoxifen (20-40 mg)
HES, glycogen and dextran with tamoxifen (20-40 mg)
HES with 4-hydroxytamoxifen
Glycogen with 4-hydroxytamoxifen
Dextran with 4-hydroxytamoxifen
HES, glycogen and dextran with 4-hydroxytamoxifen

Similarly two or more of the active agents could be present with one of the three polysaccharides or with the combination of polysaccharides. It is believed that the combination of multiple active agents could lead to synergistic effects.
HES with citicoline and alpha-tocopherol
Glycogen with citicoline and alpha-tocopherol
Dextran with citicoline and alpha-tocopherol
HES, glycogen and dextran with citicoline and alpha-tocopherol
HES with Cerovive and alpha-tocopherol
Glycogen with Cerovive and alpha-tocopherol
Dextran with Cerovive and alpha-tocopherol
HES, glycogen and dextran with Cerovive and alpha-tocopherol
HES with alpha-tocopherol and zeaxanthin
Glycogen with alpha-tocopherol and zeaxanthin
Dextran with alpha-tocopherol and zeaxanthin
HES, glycogen and dextran with alpha-tocopherol and zeaxanthin
HES with alpha-tocopherol and a flavonoid
Glycogen with alpha-tocopherol and a flavonoid
Dextran with alpha-tocopherol and a flavonoid
HES, glycogen and dextran with alpha-tocopherol and a flavonoid
HES with beta-carotene (5000 IU) and lutein (6 mg)
Glycogen with beta-carotene (5000 IU) and lutein (6 mg)
Dextran with beta-carotene (5000 IU) and lutein (6 mg)
HES, glycogen and dextran with beta-carotene (5000 IU) and lutein (6 mg)
HES with lutein and lycopene (10-20 mg)
Glycogen with lutein and lycopene (10-20 mg)
Dextran with lutein and lycopene
HES, glycogen and dextran with lutein and lycopene

Compositions containing other active chemicals described above can be prepared similarly in HES, glycogen, dextran or a mixture thereof.

The aforementioned compositions can further contain at least one antioxidant selected from the group consisting of superoxide dismutase, glutathione peroxidase, catalase, hydroxyethyl rutoside, cyclic adenosine monophosphate and vitamin C. Exemplar compositions are provided as follows.

HES with citicoline and vitamin C

Glycogen with citicoline and vitamin C

Dextran with citicoline and vitamin C

HES, glycogen and dextran with citicoline and vitamin C

The combinations of the above groups/compounds constitute different cocktails for intravenous use.

The compositions are prepared using 5-15% HES, 5-15% glycogen or 5-15% dextran again dependent on the clinical indications. When using two polysaccharides they can be used in a ratio of from 4:1 to 1:4. The compositions are always introduced intravenously. Treatment can be repeated as indicated.

The storage and delivery methods of the compositions of the invention can be any of the following,
1. Standard Mixture Delivery, wherein the polysaccharides (HES, and/or dextran, and/or glycogen) are in a mixture with the protective agents in a solution within an I.V. bag for storage and administration to a patient,
2. Piggyback Delivery, wherein the solution containing polysaccharides and the solution containing protective agents are in different compartments of the same I.V. bag and will only mix during administration when the I.V. bag is connected to the reservoir where each portion drip into after leaving their respective compartments in the bag. The mixed solution then flows from the reservoir into the bloodstream of the patient, and
3. Just-in-time Combination Delivery, wherein the polysaccharides and the protective agents are in the same I.V. bag, but in separate compartments isolated by a separating means (such as a seal, a rupturable membrane or any other known means) and will only mix during administration when the separating means is disabled between the two compartments, allowing the polysaccharides and the protective agents to become a mixture before exiting the I.V. bag.

While the compositions disclosed in this invention can be used after the onset of the diseases for treatment, they can also be used prophylactically to prevent the diseases from taking place or reducing the severity of the diseases at onsets.

1. Method of treating a human subject to prevent leakage of serum proteins from capillary endothelial junctions while simultaneously preventing the harmful effect of free radicals on cellular membranes and other organelles during a period of increased capillary permeability which comprises administering to a subject in need of such treatment an effective amount of a composition comprising at least one polysaccharide selected from the group consisting of hydroxyethyl starch, glycogen and dextran and at least one active agent selected from the group consisting of hemoglobin, polysaccharide-conjugated hemoglobin, dehydroascorbic acid, von Willebrand factor, Cerovive, citicoline, poly(ADP-ribose) polymerase inhibitor, oxidant detoxification catalyst, adenosine 2a (A2a) receptor agonist, adenosine 1 (A1) receptor agonist, adenosine, inosine, xanthin oxidase inhibitor, polyethylene-glycol-modified albumin, adenosine triphosphate, histamine, taurine, simvastatin, atrial natriuretic peptide, sphinogosine 1-phosphate, apyrase, secretory leukocyte protease inhibitor, antithrombin III, adrenomedullin, intravenous immunologlobulin, sodium beta-aescin, Δ²-1,2,3-triazoline and aminoalkylpyridine, aromatase inhibitors, and neuropilin-1, edaravone, dimethylthiourea, α-phenyl-N-tert-butyl nitrone, polynitroxyl albumin, and the antioxidant subgroup consisting of tocopherols, tocotrienols, carotenoids, minerals and mineral-containing organic compounds, polyphenols, lipoic acids, transition metal ion-binding proteins, melatonin, hormones, polyamines, tamoxifen and its metabolites and propofol, in admixture with a pharmaceutically acceptable liquid carrier.

2. Method according to claim 1 wherein said tocopherol is selected from the group consisting of alpha-, beta-, gamma- and delta-tocopherols.

3. Method according to claim 1 wherein said tocotrienol is selected from the group consisting of alpha-, beta-, gamma- and delta-tocotrienols.

4. Method according to claim 1 wherein said carotenoid is selected from the group consisting of alpha-carotene, beta-carotene, gamma-carotene, lycopene, lutein, beta-crytoxanthin, zeaxanthin, and astaxanthin.

5. Method according to claim 1 wherein said mineral is selected from the group consisting of zinc, magnesium and selenium.

6. Method according to claim 1 wherein said mineral-containing organic compounds is selected from selenoproteins.

7. Method according to claim 1 wherein said polyphenol is selected from the group consisting of flavonoids, phenolic acids, capsaicin and tannin.

8. Method according to claim 7 wherein said flavonoid is selected from the group consisting of anthocyanines, flavones, flavonols, flavanones and isoflavones.

9. Method according to claim 7 wherein said phenolic acid is a hydroxybenzoic acid.

10. Method according to claim 9 wherein said hydroxybenzoic acid is selected from the group consisting of gallic acid, ellagic acid and salicylic acid.

11. Method according to claim 1 wherein said lipoic acid is selected from the group consisting of alpha-lipoic acid and dihydrolipoic acid.

12. Method according to claim 1 wherein said transition metal ion-binding protein is selected from the group consisting of ceruloplasmin, heat-denatured ceruloplasmin, deferoxamine, lactoferrin and transferrin.

13. Method according to claim 1 wherein said hormone is selected from the group consisting of estrogen, thyroxine, dehydroepiandrosterone and 7alpha-hydroxy-dehydroepiandrosterone.

14. Method according to claim 1 wherein said polyamine is selected from the group consisting of cadaverine, putrescine, spermidine and spermine.

15. Method according to claim 1 wherein said tamoxifen metabolite is 4-hydroxytamoxifen.

16. Method according to claim 1 wherein said polysaccharide-conjugated hemoglobin is selected from the group consisting of HES-conjugated hemoglobin, dextran-conjugated hemoglobin and glycogen-conjugated hemoglobin.

17. Method according to claim 1 wherein said composition further contains at least one member selected from the group consisting of superoxide dismutase, glutathione peroxidase, catalase, hydroxyethyl rutoside, cyclic adenosine monophosphate and vitamin C.

18. Method according to claim 1 wherein said polysaccharide is present in said composition in amount of about 2 to about 30%.

19. Method according to claim 1 wherein said polysaccharide is present in said composition in amount of about 5 to 15%.

20. Method according to claim 1 wherein said composition is administered by intravenous injection in an amount of about 500 to 1500 milliliters per treatment.

21. Method of treating a human subject to prevent leakage of serum proteins from capillary endothelial junctions during a period of increased capillary permeability and simultaneously preventing the harmful effects of free radicals on cellular membranes and other organelles which comprises intravenously administering to a subject in need of such treatment an effective amount of composition comprising:
a) at least one polysaccharide selected from the group consisting of hydroxyethyl starch, glycogen and dextran, and
b) at least one active agent selected from the group consisting of hemoglobin, polysaccharide-conjugated hemoglobin, dehydroascorbic acid, von Willebrand factor, Cerovive, edaravone, dimethylthiourea, citicoline, poly(ADP-ribose) polymerase inhibitor, oxidant detoxification catalyst, adenosine 2a (A2a) receptor agonist, adenosine 1 (A1) receptor agonist, adenosine, inosine, xanthin oxidase inhibitor, polyethylene-glycol-modified albumin, adenosine triphosphate, histamine, taurine, simvastatin, atrial natriuretic peptide, sphinogosine 1-phosphate, apyrase, secretory leukocyte protease inhibitor, antithrombin III, adrenomedullin, intravenous immunologlobulin, sodium beta-aescin, Δ²-1,2,3-triazoline and aminoalkylpyridine, aromatase inhibitors, and neuropilin-1, polynitroxyl albumin, α-phenyl-N-tert-butyl nitrone and the antioxidant subgroup consisting of tocopherols, tocotrienols, carotenoids, minerals and mineral-containing organic compounds, polyphenols, lipoic acids, transition metal ion-binding proteins, melatonin, hormones, polyamines, tamoxifen and its metabolites and propofol,
in admixture with a pharmaceutically acceptable liquid carrier selected from the group consisting of 0.9% saline, 5% dextrose and Ringer's lactate and wherein said polysaccharide is present in an amount of about 2 to 30%.

22. Method according to claim 21 wherein said composition further contains at least one member selected from the group consisting of superoxide dismutase, glutathione peroxidase, catalase, hydroxyethyl rutoside, cyclic adenosine monophosphate and vitamin C.

23. A composition for treating a human subject to prevent leakage of serum proteins from capillary endothelial junctions while simultaneously preventing the harmful effect of free radicals on cellular membranes and other organelles during a period of increased capillary permeability which comprises at least one polysaccharide selected from the group consisting of hydroxyethyl starch, glycogen and dextran and at least one active agent selected from the group consisting of hemoglobin, polysaccharide-conjugated hemoglobin, dehydroascorbic acid, von Willebrand factor, Cerovive, edaravone, dimethylthiourea, citicoline, poly(ADP-ribose) polymerase inhibitor, oxidant detoxification catalyst, adenosine 2a (A2a) receptor agonist, adenosine 1 (A1) receptor agonist, adenosine, inosine, xanthin oxidase inhibitor, polyethylene-glycol-modified albumin, adenosine triphosphate, histamine, taurine, simvastatin, atrial natriuretic peptide, sphinogosine 1-phosphate, apyrase, secretory leukocyte protease inhibitor, antithrombin III, adrenomedullin, intravenous immunologlobulin, sodium beta-aescin, Δ²-1,2,3-triazoline and aminoalkylpyridine, aromatase inhibitors, and neuropilin-1, polynitroxyl albumin, α-phenyl-N-tert-butyl nitrone and the antioxidant subgroup consisting of tocopherols, tocotrienols, carotenoids, minerals and mineral-containing organic compounds, polyphenols, lipoic acids, transition metal ion-binding proteins, melatonin, hormones, polyamines, tamoxifen and its metabolites and propofol.

24. Composition according to claim 23 wherein said composition further contains at least one member selected from the group consisting of superoxide dismutase, glutathione peroxidase, catalase, hydroxyethyl rutoside, cyclic adenosine monophosphate and vitamin C.

25. A composition for treating a human subject to prevent leakage of serum proteins from capillary endothelial junctions while simultaneously preventing the harmful effect of free radicals on cellular membranes and other organelles during a period of increased capillary permeability which comprises
a) at least one polysaccharide selected from the group consisting of hydroxyethyl starch, glycogen and dextran, and
b) at least one active agent selected from the group consisting of hemoglobin, polysaccharide-conjugated hemoglobin, dehydroascorbic acid, von Willebrand factor, Cerovive, citicoline, poly(ADP-ribose) polymerase inhibitor, oxidant detoxification catalyst, adenosine 2a (A2a) receptor agonist, adenosine 1 (A1) receptor agonist, adenosine, inosine, xanthin oxidase inhibitor, polyethylene-glycol-modified albumin, adenosine triphosphate, histamine, taurine, simvastatin, atrial natriuretic peptide, sphinogosine 1-phosphate, apyrase, secretory leukocyte protease inhibitor, antithrombin III, adrenomedullin, intravenous immunologlobulin, sodium beta-aescin, Δ²-1,2,3-triazoline and aminoalkylpyridine, aromatase inhibitors, and neuropilin-1, edaravone, dimethylthiourea, polynitroxyl albumin, α-phenyl-N-tert-butyl nitrone and the antioxidant subgroup consisting of tocopherols, tocotrienols, carotenoids, minerals and mineral-containing organic compounds, polyphenols, lipoic acids, transition metal ion-binding proteins, melatonin, hormones, polyamines, tamoxifen and its metabolites and propofol,
in admixture with a pharmaceutically acceptable liquid carrier selected from the group consisting of 0.9% saline, 5% dextrose and Ringer's lactate and wherein said polysaccharide is present in an amount of about 2 to 30%.

26. Composition according to claim 25 wherein said composition further contains at least one member selected from the group consisting of superoxide dismutase, glutathione peroxidase, catalase, hydroxyethyl rutoside, cyclic adenosine monophosphate and vitamin C.

27. Method of delivering said composition according to claim 25 which comprises holding said polysaccharide and said active agent in a solution of said liquid carrier in a container, and infusing the mixture to the body of said human subject.

28. Method of delivering said composition according to claim 25 which comprises holding said polysaccharide in a solution of said liquid carrier in a first container, holding said active agent in a solution of said liquid carrier in a second container, emptying said first and second containers simultaneously into a common reservoir for mixing, and infusing the mixture in the reservoir to the body of said human subject.

29. Method of delivering said composition according to claim 25 which comprises holding said polysaccharide in a solution of said liquid carrier in a first compartment in a container, holding said active agent in a solution of said liquid carrier in a second compartment of said container, the first and second compartments being isolated by a separating means, disabling said separating means to allow said polysaccharide solution and said active agent solution to mix in said container, and infusing the mixture to the body of said human subject.

30. Method of treating a human subject according to claim 1 to prevent leakage of serum proteins from capillary endothelial junctions during a period of increased capillary permeability and simultaneously preventing the harmful effects of free radicals on cellular membranes and other organelles which comprises intravenously administering to a subject in need of such treatment said composition, wherein said treatment is performed prophylactically.

31. Method of treating a human subject according to claim 21 to prevent leakage of serum proteins from capillary endothelial junctions during a period of increased capillary permeability and simultaneously preventing the harmful effects of free radicals on cellular membranes and other organelles which comprises intravenously administering to a subject in need of such treatment said composition, wherein said treatment is performed prophylactically.
